SEQUENCE LISTING (15 sequences)

SEQ ID NO: 1 - DNA, 900 bp, Bacteriophage lambda
tatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaacc

SEQ ID NO: 2 - DNA, 26 bp, Artificial
  Primer Oligonucleotide
ctagctagctatcttgctgaactgtc

SEQ ID NO: 3 - DNA, 26 bp, Artificial
  Primer Oligonucleotide
gagaccggtggtttagccatcatctt

SEQ ID NO: 4 - DNA, 70 bp, Artificial
  Artificial Oligonucleotide
ggctggacccaacctgcgacgtccagatgaggccatctcaagcttggtagcctgttgggctagctagtag

SEQ ID NO: 5 - DNA, 78 bp, Artificial
  Artificial Oligonucleotide
ctactaaccggtccaacaggctaccaagcctgagatggcctcatctggacgtcgcaggttggacggagctacgcaggc

SEQ ID NO: 6 - DNA, 70 bp, Artificial
  Artificial Oligonucleotide
ccgaccgagcaacctgcgacgtccagatgaggccatctcaagcttggtagcctgttgggctagctagtag

SEQ ID NO: 7 - DNA, 78 bp, Artificial
  Artificial Oligonucleotide
ctactaaccggtccaacaggctaccaagcttgagatggcctcatctggacgtcgcaggttgctggaggtggagggacg

SEQ ID NO: 8 - DNA, 1024 bp, Artificial
  Artificial Sequence of a second polynucleotide complementary to a unit on the first polynucleotide representing “0”
ggctggacccaacctgcgacgtccagatgaggccatctcaagcttggtagcctgttggtatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccccaacaggctaccaagcttgagatggcctcatctggacgtcgcaggttggacggagctacgcaggc

SEQ ID NO: 9 - DNA, 1024 bp, Artificial
  Artificial Sequence of a second polynucleotide complementary to a unit on the first polynucleotide representing “1”
ccgaccgagcaacctgcgacgtccagatgaggccatctcaagcttggtagcctgttggtatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccccaacaggctaccaagcttgagatggcctcatctggacgtcgcaggttgctggaggtggagggacg

SEQ ID NO: 10 - DNA, 26 bp, Artificial
  Oligonucleotide Primer
ggaagatctggctggacgcaacctgc

SEQ ID NO: 11 - DNA, 26 bp, Artificial
  Oligonucleotide Primer
cccatcgatgcctgcgtagctccgtg

SEQ ID NO: 12 - DNA, 26 bp, Artificial
  Oligonucleotide Primer
ggaagatctccgaccgagcaacctgc

SEQ ID NO: 13 - DNA, 26 bp, Artificial
  Oligonucleotide Primer
cccatcgatcgtccctccacctccag

SEQ ID NO: 14 - DNA, 17 bp, Artificial
  Oligonucleotide Primer
tccagttagctcaaggg

SEQ ID NO: 15 - DNA, 17 bp, Artificial
  Oligonucleotide Primer
gagattaagtgcgcgcc

(19) United States
(12) Patent Application Publication
(10) Pub. No.: US 2011/0117544 A1
(43) Pub. Date: May 19, 2011

(54) METHOD FOR PRODUCING AN AMPLIFIED POLYNUCLEOTIDE SEQUENCE

(75) Inventor: Preben Lexow, Oslo (NO)

(73) Assignee: LINGVITAE AS, Oslo (NO)

(21) Appl. No.: 11/817,187

(22) PCT Filed: Mar. 1, 2006
(86) PCT No.: PCT/GB2006/000711

§ 371 Date: May 28, 2008

(30) Foreign Application Priority Data
Mar. 1, 2005 (GB) 0504184.3

(51) Int. Cl. C12Q 1/68; C07H 21/00

(52) U.S. Cl. 435/6; 536/25.3

(57) ABSTRACT

There is a method for converting at least a part of a target polynucleotide sequence into a different sequence, comprising the steps of: (i) hybridising two or more first polynucleotides to adjacent positions on the target and linking first polynucleotides together, wherein at least one of the first polynucleotides comprises at least one nucleotide that is not complementary to the target; and (ii) dissociating the hybrid of step (i) and forming a second polynucleotide hybridised to the first polynucleotide, the second polynucleotide comprising at least one portion complementary to a portion on the first polynucleotide and comprising the complement to the at least one nucleotide of step (i).

### FIELD OF THE INVENTION

This invention relates to methods for magnifying the sequence information contained within a polynucleotide.

### BACKGROUND TO THE INVENTION

Advances in the study of molecules have been led, in part, by improvement in technologies used to characterise the molecules or their biological reactions. In particular, the study of the nucleic acids DNA and RNA has benefited from developing technologies used for sequence analysis and the study of hybridisation events.

The principal method in general use for large-scale DNA sequencing is the chain termination method. This method was first developed by Sanger and Coulson (Sanger et al., Proc. Natl. Acad. Sci. USA, 1977; 74: 5463-5467), and relies on the use of dideoxy derivatives of the four nucleotides which are incorporated into the nascent polynucleotide chain in a polymerase reaction. Upon incorporation, the dideoxy derivatives terminate the polymerase reaction and the products are then separated by gel electrophoresis and analysed to reveal the position at which the particular dideoxy derivative was incorporated into the chain.

Although this method is widely used and produces reliable results, it is recognised that it is slow, labour-intensive and expensive.

U.S. Pat. No. 5,302,509 discloses a method to sequence a polynucleotide immobilised on a solid support. The method relies on the incorporation of 3′-blocked bases A, G, C and T having a different fluorescent label to the immobilised polynucleotide, in the presence of DNA polymerase. The polymerase incorporates a base complementary to the target polynucleotide, but is prevented from further addition by the 3′-blocking group. The label of the incorporated base can then be determined and the blocking group removed by chemical cleavage to allow further polymerisation to occur. However, the need to remove the blocking groups in this manner is time-consuming and must be performed with high efficiency.

WO-A-00/39333 describes a method for sequencing a polynucleotide by converting the sequence of a target polynucleotide into a second polynucleotide having a defined sequence and positional information contained therein. The sequence information of the target is said to be “magnified” in the second polynucleotide, allowing greater ease of distinguishing between the individual bases on the original target molecule. This is achieved using “magnifying tags” which are predetermined nucleic acid sequences. Each of the bases adenine, cytosine, guanine and thymine on the target molecule is represented by an individual magnifying tag, converting the original target sequence into a magnified signal or sequence. Conventional techniques may then be used to determine the order of the magnifying tags, and thereby determine the specific sequence on the target polynucleotide.

However, although useful, there is still a need to provide further methods for the production of the “magnified sequences”, to improve the flexibility in the design of the magnified sequences.

### SUMMARY OF THE INVENTION

The present invention is based on the realisation that sequence information provided on a target polynucleotide can be converted into a different sequence by producing a polynucleotide representing modified characteristics of the target polynucleotide.

According to a first aspect of the invention, a method for converting at least a part of a target polynucleotide sequence comprises the steps of:

(i) hybridising two or more first polynucleotides to adjacent positions on the target and linking two or more first polynucleotides together, wherein at least one of the first polynucleotides comprises at least one nucleotide that is not complementary to the target; and

(ii) dissociating the hybrid of step (i) and forming a second polynucleotide hybridised to the first polynucleotide, the second polynucleotide comprising at least one portion complementary to a portion on the first polynucleotide and comprising the complement to the at least one nucleotide of step (i).

The invention permits the creation of massively magnified sequence information, which allows sequence data to be read by read-out platforms which cannot read the smaller tags currently seen in the art. The invention also permits modifications to be made to the target polynucleotide sequence, to correct sequence errors introduced into the target during its synthesis.

### DESCRIPTION OF THE DRAWINGS

The invention is described with reference to the following drawings, wherein:

FIG. 1 is a schematic representation of the conversion of a target polynucleotide into an amplified second polynucleotide, wherein a binary labeling system is used to represent the bases in the target polynucleotide as a series of “0”s and “1”s in the target and second polynucleotides and wherein the second polynucleotide is the complement of the linked first polynucleotides; and

FIG. 2 is a schematic of one embodiment of a first polynucleotide, comprising terminal regions complementary to a unit on the target polynucleotide separated by a stem-loop region, the loop region is taken from lambda DNA and the SEQ ID NOs. refer to primers used in the Example.

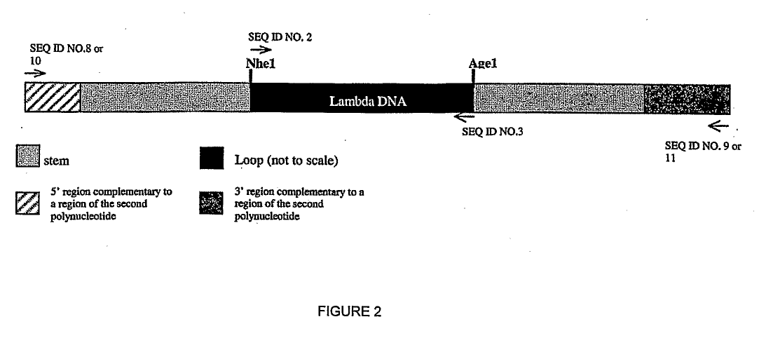

### DESCRIPTION OF THE INVENTION

The methods of the invention rely on the conversion of specific characteristics of a target molecule into a series of first polynucleotides having distinct, defined, units of nucleic acid sequence designed for various purposes, including the amplification of the sequence of the target polynucleotide or to introduce additional nucleic acid sequences which are useful in the characterisation or modification of the resulting (second) polynucleotide.

The first polynucleotides each have a distinct, defined, nucleic acid sequence that includes a sequence complementary to a distinct unit of the target polynucleotide, thereby permitting hybridisation to occur. The first polynucleotides comprise the same or a larger number of nucleic acids (bases) than the corresponding unit of the target polynucleotide. Each first polynucleotide will usually be more amplified (in terms of number of nucleotides in the resulting polynucleotide) than the target polynucleotide and the amplified regions may represent a particular characteristic on the target molecule. The first polynucleotides are then linked to form a polynucleotide of defined sequence.

The term “polynucleotide” is well known in the art and is used to refer to a series of linked nucleic acid molecules, e.g. DNA or RNA. Nucleic acid mimics, e.g. PNA, LNA (locked nucleic acid) and 2′-O-methRNA are also within the scope of the invention.

The reference to the bases A, T(U), G and C, relate to the nucleotide bases adenine, thymine (uracil), guanine and cytosine, as will be appreciated in the art. Uracil replaces thymine when the polynucleotide is RNA, or it can be introduced into DNA using dUTP, again as well understood in the art.

The target polynucleotide will preferably comprise distinct “units” of nucleic acid sequence, as described by WO-A-00/39333, which is incorporated herein by reference. Each distinct and predefined unit, or unique combination of units represents a sequence or characteristic of an earlier studied molecule. The target polynucleotide is therefore a “design polymer” in the sense that its sequence represents the characteristics of the earlier molecule. Each unit will preferably comprise two or more nucleotide bases, preferably from 2 to 50 bases, more preferably 2 to 20 bases and most preferably 4 to 10 bases, e.g. 6 bases. There are at least two different bases contained in each unit and the sequence of these represents a characteristic of the earlier molecule. In a preferred embodiment there are three different bases in each unit. The design of the units is such that it will be possible to distinguish the different units during a “read-out” step.

The “magnifying tag” method as disclosed in WO-A-00/39333 may be used to create the target polynucleotide. This method relates to the sequencing of polynucleotides by converting the sequence of an original polynucleotide into a further polynucleotide having a defined sequence and (optionally) positional information contained therein. The sequence information of the original polynucleotide is said to be “magnified” in the further polynucleotide, allowing greater ease of distinguishing between the individual bases on the original polynucleotide. This is achieved using “magnifying tags” which are predetermined nucleic acid sequences. Each of the bases adenine, cytosine, guanine and thymine (uracil) on the original polynucleotide is represented by an individual magnifying tag, converting the original sequence into a magnified sequence. It is this magnified sequence that may be taken as the “target” polynucleotide for further conversion using the methods of the invention.

This “magnifying tag” technique can be used to convert the characteristics of any molecule into a magnified sequence. For example, a polypeptide may be converted into a magnified polynucleotide sequence. This magnified polynucleotide may also be taken as the “target” polynucleotide using the methods of the invention.

The “magnifying tag” method can also be used to determine the absolute or relative amounts of a unique target molecule in a sample, and to track individual molecules within a population, as described in co-pending International Patent Application No. PCT/GB2005/000218, the content of which is incorporated herein by reference. A magnified sequence created using this method may also be taken as the “target” polynucleotide using the methods of the invention.

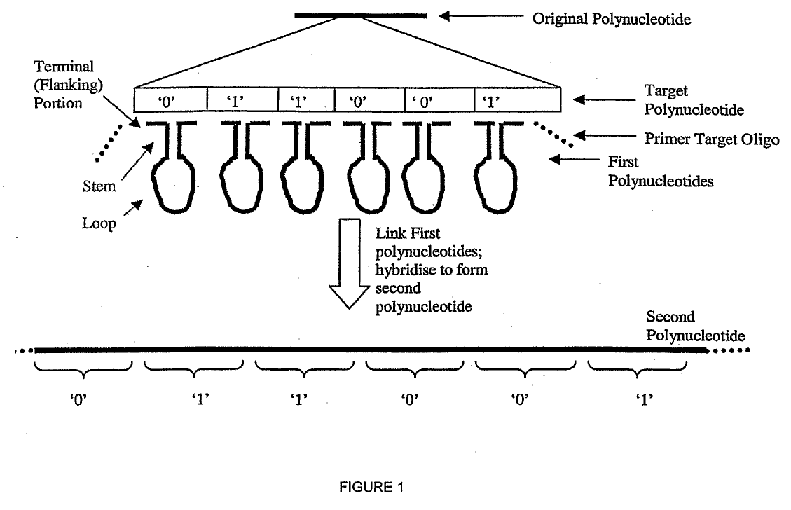

In a preferred embodiment, the target polynucleotide contains binary sequence information, as described in WO-A-00/39333 and WO-A-04/094664, each of which are hereby incorporated by reference. As there are only two different sequences in the target polynucleotide, only two different first polynucleotides are required, i.e. one that hybridises to the sequence encoding “0” and one that hybridises to the sequence encoding “1”, for example as illustrated in FIG. 1. The second polynucleotide will therefore contain a series of “0” and “1” signals, that represent the characteristics of the target. For example, where the target is a polynucleotide, adenine is represented by “0”+“0”, cytosine by “0”+“1”, guanine by “1”+“0” and thymine by “1”+“1”.

Each first polynucleotide may also comprise a distinct “unit” or combination of units of nucleic acid sequence that represents a specific unit of the target polynucleotide. In this embodiment, each first polynucleotide unit comprises at least one more base (nucleotide) than the corresponding target polynucleotide unit, preferably 3 or more nucleotide bases, more preferably from 3 to 1000 bases, yet more preferably 100 to 1000 bases. The purpose of this embodiment is simply to amplify the size of the sequence. By “amplify” it is meant that the number of bases is increased. Each first polynucleotide may comprise at least two portions, one complementary to at least a portion of a unit of the target polynucleotide to allow specific hybridisation with its complement, and a second portion which comprises the at least one additional base. Preferably, each first polynucleotide comprises three portions: two flanking portions complementary to a unit of the target polynucleotide and a third intervening portion comprising the additional base(s). The two flanking portions hybridise to a unit on the target polynucleotide, and the intervening portion forms a loop representing the “amplified” sequence. This is illustrated in FIG. 1. The portion that comprises the additional base(s) may be designed so that the additional base(s) represent one or more specific bases on the target polynucleotide, i.e. the first polynucleotide is a “magnified” sequence of the target polynucleotide. In this embodiment, each base in the target polynucleotide to which the first polynucleotide hybridises (by complementary base pairing between the flanking portions of the first polynucleotide and a unit of the target polynucleotide) is represented by a defined sequence in the intervening portion of the first polynucleotide. The intervening portion therefore contains a series of magnified signals, as described in WO-A-00/39333. It will be appreciated that if the unit on the target polynucleotide is already a magnified signal, this embodiment allows further magnification.

Alternatively, the additional base(s) in the first polynucleotide may be used to provide sequences for the modification of the first polynucleotide (e.g. sites for primer hybridisation for subsequent amplification using PCR). In another alternative, the additional bases may be of any sequence, known or unknown, acting purely as a spacer to separate the “units” of the target polynucleotide so that the read-out step can be performed with increased distance and therefore enhanced discrimination between individual units.

To incorporate a large number of bases into the portion that contains the additional bases, it is preferred that the first polynucleotide is designed to form a stem and loop structure. Stem-loop structures (often referred to as hairpin loops) are well known in the art to be single-stranded polynucleotides that are at least partially self-complementary, and can therefore self-hybridise. These self-complementary bases form a double-stranded portion known as the “stem”. Non-self-complementary bases form the “loop” at the end of the stem.

In a different embodiment, the first polynucleotides may be designed to correspond to the expected sequence for the target polynucleotide. In this embodiment, the first polynucleotide may be used to “clean up”, or correct errors in, the sequence of the target polynucleotide, prior to analysis. For example, if the target polynucleotide is constructed to correspond to specific bases on an original polynucleotide, but contains minor errors in the resulting sequence, the first polynucleotide can be used to correct mistakes in the target polynucleotide prior to analysis of the sequence. In this embodiment, a first polynucleotide is designed to hybridise to the correct, expected or desired target sequence. If a small number of errors exist in the target polynucleotide, for example introduced in the conversion from the original polynucleotide, the first polynucleotide and target will not be 100% complementary. However, the first polynucleotide will still hybridise to the target polynucleotide. It will be apparent to one skilled in the art that hybridisation conditions can be optimised to allow hybridisation of sequences that are not 100% complementary. The first polynucleotide can therefore be used to correct errors in the target or, conversely, to enter small changes (errors) into the sequence. This corrected or changed sequence will be maintained in the second polynucleotide, i.e. the complement of the linked first polynucleotides.

This technique can also be applied when larger changes need to be introduced into the sequence, so that the first polynucleotide has a different sequence to the target polynucleotide. It will be apparent that there must be sufficient complementarily between the two sequences for hybridisation to occur. For successful ligation between 2 first polynucleotides to occur, it is preferred that the terminal nucleotide, at least, in each of the first polynucleotides that are to be ligated, are hybridised to the target polynucleotide. This embodiment is particularly useful for introducing sequences that may be required in subsequence analysis or manipulation of the sequences. For example, certain sequences may be suitable for polymerase amplification but less suitable in the read-out phase. The ability to “switch” the sequences in the manner described above allows both to be performed optionally.

The second polynucleotide is the complement of the first polynucleotides; any sequence change between the target and first polynucleotides will therefore be retained in the second polynucleotide.

Any of the 3 main embodiments of first polynucleotides, i.e. amplification, error removal and sequence switching, can be combined. For example, magnification of the target polynucleotide can be done simultaneously with error removal and/or sequence switching. The design of the first polynucleotide will depend on their purpose, as will be apparent to one skilled in the art.

Any method may be used to create the first polynucleotides; suitable molecular cloning and oligonucleotide synthesis techniques will be apparent to the skilled person. A preferred method is to create the first polynucleotide from a number of separate oligonucleotide components, the separate components being connected by molecular cloning to form the first polynucleotide. This is particularly useful when creating the preferred stem-loop first oligonucleotides for the amplification embodiment. In this embodiment, three separate oligonucleotide components may be used. Preferably, a first and a second component each comprise a region complementary to a distinct region of a unit on the target polynucleotide, and a “stem” region that is not complementary to the unit on the target polynucleotide. The “stem” sequence in the first oligonucleotide component is complementary to the “stem” sequence in the second oligonucleotide component. These two components will form the regions of the first polynucleotide that can hybridise to the target polynucleotide, while the “stem” sequences will interact to form a double-stranded stem region. These first and second oligonucleotide components may be referred to as “stem-containing oligonucleotides”. The third oligonucleotide component is a “loop” sequence, which is cloned onto the ends of the stem sequences, thereby forming a first polynucleotide comprising a stem-loop structure flanked by two regions complementary to a unit on the target polynucleotide.

It will be appreciated that, in order for molecular cloning techniques to be used, the first polynucleotide should preferably be double-stranded and, if the first polynucleotide is created using cloning, the oligonucleotide components used in the production of the first polynucleotide should be double-stranded. Each stem-containing oligonucleotide component is therefore designed as single-stranded. This single-stranded stem-containing oligonucleotide may be considered as a “sense” strand. An oligonucleotide that is complementary to this is also designed, which is the “anti-sense” strand. These “sense” and “anti-sense” oligonucleotides can then be hybridised together to form a double-stranded polynucleotide that can be used in molecular cloning procedures, as illustrated in FIG. 1.

Preferably, the oligonucleotides are designed to contain restriction enzyme sites, for ease of cloning.

The two double-stranded stem-containing oligonucleotides are ligated to the loop sequence. Molecular cloning strategies, preferably using a restriction-enzyme based strategy, will be apparent to one skilled in the art. To ensure the correct orientation and construction of the stem-loop structure, the two stem-containing double-stranded oligonucleotides must be attached to opposite ends of the loop sequence.

Any loop sequence may be used. Preferably, the loop comprises a plurality of residues, for example between 2 and 1000 residues. The size of the loop can be varied depending on the amount of signal amplification required. The loop sequence may be random, or taken from any source, for example, the loop sequence may be taken from the bacteriophage lambda as shown as SEQ ID NO. 1. For use in molecular cloning, it is preferred that the loop sequence is double-stranded.

The ligation of the two stem-containing oligonucleotides (which contain the regions complementary to a unit on the target polynucleotide) to the loop region creates a double-stranded polynucleotide. One strand of this, the “sense” strand, is the first polynucleotide suitable for hybridisation to a unit of the target polynucleotide. This contains the first and second portions complementary to a unit on the target polynucleotide, and the third stem-loop portion. Although a single-stranded first polynucleotide is eventually required, it is preferable to create a double-stranded polynucleotide for ease of manipulation during cloning procedures. In order to obtain large quantities of this double-stranded polynucleotide, it can be amplified by the polymerase reaction and cloned into a vector. Suitable cloning strategies will be apparent to a skilled person, and any suitable vector may be used. This allows the production of large amounts of the polynucleotide in recombinant systems such as E. coll. In order to produce the double-stranded polynucleotide from this construct, a polymerase reaction is carried out using primers that specifically amplify the double-stranded polynucleotide containing the first polynucleotide.

Other methods may be used to create large quantities of the double-stranded polynucleotide, for example a linear polymerase reaction may be performed. Alternatively, it may not be necessary to further amplify the double-stranded polynucleotide.

Once the double-stranded polynucleotide is obtained, it is necessary to remove the anti-sense strand to produce the single-stranded first polynucleotide suitable for hybridising to the target polynucleotide. In a preferred embodiment, an exonuclease reaction is used that will degrade the anti-sense strand only.

Removal of the anti-sense strand yields the single-stranded first polynucleotide, which may be contacted with the target polynucleotide. However, due to the secondary structure (the stem-loop), it is preferred that the first polynucleotide is subjected to specific folding conditions prior to use, to ensure that it is folded correctly. A suitable folding procedure is heating to 95° C. to remove any residual 2° structure, before cooling to 20° C. in 0.1° C. increments.

To perform the method of the invention, the target polynucleotide is contacted with at least two first polynucleotides, under hybridising conditions. An example of such conditions is incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

After hybridisation, each unit of the target polynucleotide has a first polynucleotide hybridised to it (see FIG. 1). The target polynucleotides may be contacted with the first polynucleotide simultaneously or sequentially. The first polynucleotides are then joined and disassociated from the target polynucleotide. At least two first polynucleotides are hybridised to the target polynucleotide and joined together. In one embodiment, at least three polynucleotides are hybridised to the target and joined together. The second polynucleotide is created by hybridisation to the linked first polynucleotides to form a single continuous second polynucleotide. The first polynucleotides may be joined by any suitable method, chemical or enzymatic. Preferably, the first polynucleotides are ligated, using a ligase enzyme. It will be apparent to one skilled in the art that ligation of two first polynucleotides requires that they hybridise immediately adjacent to one another. The second polynucleotide is the complement of the linked first polynucleotides, which contains a series of defined sequences which represent, ultimately, the characteristics on the target molecule.

The second polynucleotide may then be analysed. Any analysis procedure suitable for nucleic acids may be used. Preferably, the analysis procedure comprises a sequencing procedure. Sequencing methods are well known in the art.

The design of the first polynucleotides is such that it will be possible to distinguish each different unit in the second polynucleotide during a “read-out” step, involving either the incorporation of detectably labeled nucleotides in a polymerisation reaction, or on hybridisation of complementary oligonucleotides.

Since the regions of the first polynucleotide (and therefore the second polynucleotide) which hybridised to the target polynucleotide are, in a preferred embodiment, complementary to the magnified tags of the target polynucleotides, these regions may also be magnified tags.

The amount of second polynucleotide can optionally be increased by a polymerase reaction. Primers may be used that are complementary to regions of the second polynucleotide, preferably terminal regions, as will be appreciated by one skilled in the art. In a preferred embodiment, specific primer target oligonucleotides are incorporated at the 5′ and 3′ termini of the second polynucleotide. These sequences may be incorporated into the terminal first polynucleotides or may be added as separate oligonucleotides and ligated into the second polynucleotide. Preferably, these primer target sequences are unique in the second polynucleotide. Oligonucleotide primers for use in the polymerase reaction can then be designed to hybridise to these primer target sequences, allowing polymerase amplification of the entire second polynucleotide.

A polymerase reaction to amplify the second polynucleotide will result in a double-stranded polynucleotide molecule. Due to the possibility of secondary structure of the single-stranded second polynucleotide, it is preferred that a strand-displacing polymerase is used in the polymerase reaction. Strand-displacing polymerases are well known in the art.

The invention is further illustrated by the following example.

### EXAMPLE

(1) Cloning Strategy Used to Produce (Double-Stranded) First Polynucleotides Complementary to “0” and “1” Units in the Second Polynucleotide.

The loop-sequence (SEQ ID NO. 1) was amplified from the bacteriophage lambda genome using primers 213 (SEQ ID NO. 2) and 214 (SEQ ID NO. 3) in a polymerase reaction. These primers contain the restriction sites Nhe1 and Age1 respectively in their 5′ tails. The polymerase reaction product was then digested using the restriction enzymes Nhe1 and Age1.

A stem-containing oligonucleotide containing a sequence complementary to a 5′ portion of the unit on the first polynucleotide representing “0”, and a separate stem sequence, was designed (see SEQ ID NO. 4). The complement (“anti-sense”) to this sequence was designed and annealed. This method was repeated to create a stem-containing oligonucleotide that contains a sequence complementary to a 3′ portion of the unit on the target polynucleotide representing “0”. The resulting double-stranded oligonucleotides are designated herein as 5′(0) (SEQ ID NO. 4) and 3′(0) (SEQ ID NO. 5). The same method was then used to create oligonucleotides complementary to the “1” unit, 5′(1) (SEQ ID NO. 6) and 3′(1) (SEQ ID NO. 7).

5′ (0) and 5′ (1) were then digested with Nhe1; 3′ (0) and 3′(1) were digested with Age1.

5′ (0) and 3′ (0) were ligated to the digested “loop” PCR and 5′(1) and 3′(1) were ligated to the digested “loop” product in a separate reaction, thereby creating the first polynucleotides complementary to “0” and “1” (SEQ ID NOS. 8 and 9, respectively), in double-stranded form.

The ligated second polynucleotides were amplified by PCR using the primers 215/216 (SEQ ID NOS. 10 and 11, respectively) for “0” and 217/218 (SEQ ID NOS. 12 and 13, respectively) for “1”.

Optionally, the PCR product of the last step above may be cloned into pZeroBlunt (Invitrogen).
(2) Production of Single-Stranded First Polynucleotides

The double-stranded first polynucleotides (SEQ ID NO. 8 and 9) were amplified in a polymerase reaction, using VENT DNA polymerase. Five 50 μl polymerase reactions were performed in order to create enough material. An identical reaction without template served as a negative control.

Upon completion, the five reactions were pooled and the DNA precipitated by standard ethanol precipitation. The concentration of the precipitated PCR product from step 2 was measured; a dilution series of the MassRuler DNA ladder (100, 50, 25 and 5 ng of 1031 bp band) was run next to 2 μl of the precipitated sample and 2 μl of the negative control on a 0.8% agarose gel at 30V for 5 minutes in addition to 50V for 60 minutes, followed by staining with ethidium bromide. The concentration was measured using the Quantity One software from BioRad and the PCR product from step 2 diluted to 50 ng/μl in Iris buffer (10 mM).

6 μl (300 ng) of the PCR product was digested using 2 U lambda exonuclease. The anti-sense strand of the product is susceptible to degradation due to the 5′ phosphorylation.

The exonuclease reaction was incubated at 37° C. for 30 minutes before heat inactivation of the exonuclease at 75° C. for 10 minutes.

1 μl (15 ng single-stranded first polynucleotide) of the reaction mix along with 0.6 μl (30 ng) of the undigested material (from step 4) was run on a 10% TBE PAGE gel (BioRad) at 150V for 80 minutes, followed by staining with SybrGold.

The rest of the reaction mix was separated, along with 2 μl undigested PCR product (50 ng/μl ), on a 0.8% agarose gel at 30V for 5 minutes in addition to 50V for 60 minutes. The unique single-stranded band was gel purified using the Minielute spin columns from Qiagen and eluted in 30 μl buffer EB. The gel-purified single-stranded first polynucleotide was ethanol precipitated and resuspended in 8 μl water. Anticipating 10% loss during the gel purification step, this yields a concentration of 15 ng/μl single-stranded first polynucleotide.

The single-stranded first polynucleotide was then phosphorylated using T4 polynucleotide kinase, the reaction incubated at 37° C. for 30 minutes and heat inactivated at 65° C. for 10 minutes.

The phosphorylated single-stranded first polynucleotide was ethanol precipitated and resuspended in 10 μl (12 ng/μl ).

5 μl (60 ng) of phosphorylated single-stranded second polynucleotide was folded in NEB2 buffer by heating to 95° C. for one minute before cooling to 20° C. at a rate of 0.1° C./second. The formation of correctly folded 2° structure was analysed by running 4 μl (24 ng) of the folded single-stranded second polynucleotide on a 10% TEB PAGE gel at 150V for 120 minutes. 2 μl (24 ng) phosphorylated single-stranded second polynucleotide and 0.5 μl double-stranded first polynucleotide (25 ng) were included on the gel as controls.
(3) Production of the Linked First Polynucleotides

The “binary” target polynucleotide and primer target oligonucleotides were mixed in a 1:1 ratio. Twice the molar amount of each single-stranded first polynucleotide was added, together with T4 DNA ligase buffer.

This reaction mix was incubated at 50° C. for 30 minutes, followed by ramping to 37° C. at 0.1° C./second, incubating at 37° C. for 10 minutes, followed and by ramping to 20° C. at 0.1 ° C./second and holding at 20° C.

0.2 μl T4 DNA ligase (1 WeissUnit) was then added to the reaction mix and incubated at 25° C. for 60 minutes before heat inactivation at 65° C. for 10 minutes.

The ligated construct was amplified by a polymerase reaction, using a strand displacing polymerase and the primers SEQ ID NOS. 14 and 15, which hybridise to the primer target oligonucleotides incorporated into the ligated polynucleotide.

The polynucleotide was analysed on a 0.8% agarose gel at 30V for 5 minutes in addition to 50V for 45 minutes.

Table of Primers

| Name | Sequence |
|---|---|
| 213/ROC/FU/loop | 5′-ctagctagctatcttgctgaactgtc-3′ |
| | (SEQ ID NO. 2) |
| 214/ROC/RU/loop | 5′-gagaccggtggtttagccatcatctt-3′ |
| | (SEQ ID NO. 3) |
| 250\ROC\FU\5′SDPP | 5′-TCCAGTTAGCTCAAGGG-3′ |
| | (SEQ ID NO. 14) |
| 251\ROC\RU\3′SDPP | 5′-GAGATTAAGTGCGCGCC-3′ |
| | (SEQ ID NO. 15) |
| 215/ROC/FU/ESP(0) | 5′-ggaagatctggctggacgcaacctgc-3′ |
| | (SEQ ID NO. 10) |
| 216/ROC/RU/ESP(0) | 5′-cccatcgatgcctgcgtagctccgtg-3′ |
| | (SEQ ID NO. 11) |
| 217/ROC/FU/ESP(1) | 5′-ggaagatctccgaccgagcaacctgc-3′ |
| | (SEQ ID NO. 12) |
| 218/ROC/RU/ESP(1) | 5′-cccatcgatcgtccctccacctccag-3′ |
| | (SEQ ID NO. 13) |

I claim:

1: A method for converting at least a part of a target polynucleotide sequence into a different sequence, comprising:
(i) hybridising two or more first polynucleotides to adjacent positions on the target and linking first polynucleotides together, wherein at least one of the first polynucleotides comprises at least one nucleotide that is not complementary to the target; and
(ii) dissociating the hybrid of (i) and forming a second polynucleotide hybridised to the first polynucleotide, the second polynucleotide comprising at least one portion complementary to a portion on the first polynucleotide and comprising the complement to the at least one nucleotide of (i).

2: The method according to claim 1, wherein the first polynucleotide comprises a series of nucleotides that are not complementary to the target.

3: The method according to claim 1, wherein each first polynucleotide comprises one or more loop regions, each loop comprising a plurality of nucleotides that are not complementary to the target sequence.

4: The method according to claim 1, wherein the target sequence comprises one or more single nucleotide mutations, and the first polynucleotide comprises a sequence that represents the complement of a non-imitated target sequence, wherein the resulting second polynucleotide corresponds to a non-mutated target sequence.

5: The method according to claim 1, wherein the first polynucleotides are ligated together.

6: The method according to claim 1, further comprising the step of analysing the sequence of the second polynucleotide.

7: The method according to claim 3, wherein the loop regions comprise more than 5 additional bases.

8: The method according to claim 2, wherein the second polynucleotide comprises a sequence intended as a target for a primer in an amplification reaction.

9: The method according to claim 1, wherein the target polynucleotide is a synthetic polynucleotide comprising a series of defined units of sequence, each unit or specific combination of units representing a characteristic of another molecule.

10: The method according to claim 1, wherein three or-more first polynucleotides are hybridised to the target in (i).

11: The method according to claim 3, wherein the second polynucleotide comprises a sequence intended as a target for a primer in an amplification reaction.
